(19) United States
(12) Patent Application Publication
(10) Pub. No.: US 2023/0247972 A1
(43) Pub. Date: Aug. 10, 2023

(54) Method of Producing an Animal of a Particular Breed

(71) Applicant: Neighborhood Bullies LLC, Katy, TX (US)

(72) Inventors: Brandon Dixon, Katy, TX (US); John Udegbunam, Houston, TX (US)

(21) Appl. No.: 17/666,542

(22) Filed: Feb. 7, 2022

(51) Int. Cl. A01K 67/02; C12Q 1/6888

(52) U.S. Cl. CPC A01K 67/02; C12Q 1/6888; C12Q 2600/124

(57) ABSTRACT

This disclosure relates to an improved method of producing an animal of a particular breed. The method can comprise the step of determining genetic test results. The genetic test results can relate to one or more genetic characteristics of the animal. The method can also comprise the step of comparing the genetic test results to each other genetic test result of a plurality of other genetic test results to determine a probability that if the first animal is mated with an animal associated with the other genetic test result that resulting offspring would have characteristics each within a predetermined threshold. The method can also comprise the steps of determining the other animal to be a suitable match if the probability is above an acceptable probability threshold, and producing offspring with the animal and at least one of the other animals determined to be a suitable match.

### BACKGROUND

This disclosure relates to an improved method of producing an animal of a particular breed. This disclosure also relates to a method for integrating a network of household pet owners into a pet supply chain. The pet industry continues to make explosive growth as more and more people enjoy pet ownership. Since many people have an affinity for particular breeds, there is a substantial market for pure bread pets, that is pets conforming to a particular set of characteristics that define a certain breed. One reason some prefer a particular breed is that dogs within a particular breed, apart from their physical characteristics, tend to have similar dispositions to other dogs in the particular breed. For example, some dog breeds have a particularly keen sense of smell, while other breeds are naturally adept and hunting birds. As such, choosing a particular dog by its breed can be particularly beneficial to a prospective pet owner looking to own a dog with particular qualities. However, methods for providing a secure and convenient way for owning a pet of a particular breed have not improved substantially over the years.

Additional problems in breeding animals include inhumane treatment of animals that are usually being farmed on high-volume breeding facilities e.g. puppy mills. In such facilities, there is often little human interaction, and animals are kept in stacked wire cages (some milled dogs never touch grass). Also, pets produced from high-volume breeding facilities are often develop serious health and behavioral issues. These issues can be due to the cruel conditions in which these animals are bred. Small and intermediate breeders who can be capable of providing better facility and give better treatment to animals are also facing problems such as retail space or having a better platform in which breeders can conveniently market their animal’s offspring.

Furthermore, most breeding facility does not have the proper method in producing a desired breed of animal. Having a method in determining a way to find a suitable match, such as genetic sampling, for each animal being bred can help in the biodiversity in the breed of animal. This method can also help breeders in producing healthy offspring that are less susceptible to common diseases.

Affordability is another common problem faced by individuals interested in buying high standard animal. Usually, individuals would have to pay a large amount of money upfront to buy a specific breed, which can deter most buyer in purchasing a pet. Thus, having a method of being able to offer the pet through staggard payment can make ownership of a pet with a high standard breed be convenient and possible. As such it would be useful to have an improved method of producing an animal of a particular breed. It would also be useful to have a method for integrating a network of household pet owners into a pet supply chain.

### SUMMARY

This disclosure relates to an improved method of producing an animal of a particular breed. The method can comprise the step of determining genetic test results. The genetic test results can relate to one or more genetic characteristics of the animal. The method can also comprise the step of comparing the genetic test results to each other genetic test result of a plurality of other genetic test results to determine a probability that if the first animal is mated with an animal associated with the other genetic test result that resulting offspring would have characteristics each within a predetermined threshold. One or more of the predetermined thresholds can relate to a breed of such animal. The method can also comprise the steps of determining the other animal to be a suitable match if the probability is above an acceptable probability threshold, and producing offspring with the animal and at least one of the other animals determined to be a suitable match.

This disclosure also relates to a method for integrating a network of household pet owners into a pet supply chain. The method can comprise the steps of developing a network of pet owners and information of one or more household pets of each of the pet owners, and determining a suitable match of pets to produce offspring having positive characteristics in relation to a breed. Furthermore, the method can comprise the steps of obtaining permission from each of the owners of matched pets to produce offspring using the matched pets, and facilitating the breeding of the matched pets and commercialization of the offspring with a plurality of services from the following: seminal fluid collection and storage, artificial insemination, birthing, whelping, marketing of the offspring, and sale of the offspring.

### BRIEF DESCRIPTION OF THE DRAWINGS

FIG. 1 illustrates an embodiment of a pet supply chain system.

FIG. 2 illustrates profiling a pet on a pet supply chain system.

FIG. 3 illustrates an example of genetic characteristics of dog breeds.

FIG. 4 illustrates another embodiment of a pet supply chain system further comprising a pet application.

FIG. 5 illustrates a screenshot of an embodiment of a pet application.

FIG. 6 illustrates a sign-up screen.

FIG. 7 illustrates an add-pet screen.

FIG. 8 illustrates a pet owner’s screen comprising a pets list.

FIG. 9 illustrates a social network page of a pet application.

FIG. 10 illustrates an exemplary method for producing an animal breed.

FIG. 11 illustrates an exemplary method for integrating a network of household pet owners into a pet supply chain.

FIG. 12 illustrates an exemplary method for providing a scheduled pet services to pets.

### DETAILED DESCRIPTION

Described herein is an improved method of producing an animal of a particular breed as well as a method for integrating a network of household pet owners into a pet supply chain. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers’ specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

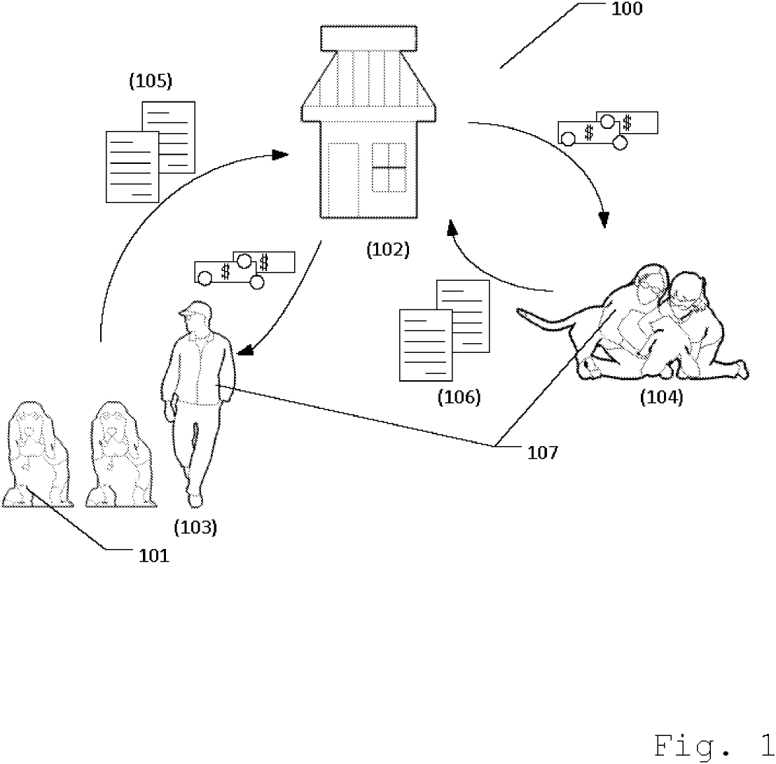

FIG. 1 illustrates an embodiment of pet supply chain system 100. In this embodiment, pet supply chain 100 provides a safe method for facilitating pet breeding to produce breeds and making them accessible to the general public. Pet supply chain system 100 can connect one or more pet stores 102 with small breeders 103 and hobby breeders 104. Pet store 102 can be a retail business that sells pets 101. For purposes of this disclosure, pet 101 is a domesticated animals such as a cats or dog. Small breeders 103 refers to individuals or small businesses (typically home businesses) that breed animals of particular breeds. For purpose of this disclosure, breed refers to a subset of a species having specific characteristics or traits. Further for purposes of this disclosure, Breed can also refer to a traditional breed such as a labrador or poodle, or a combination breed such as a labradoodle. For purposes of this disclosure, hobby breeders 104 refers to household pet owners that usually purchases an animal to care for them as pets, and can be a person who purchased the pet from pet store 102. Further, in this disclosure small breeders 103 and hobby breeders 104 can be referred to generally as pet owners 107.

In one embodiment, pets 101 can be sourced from small breeders 103. In such embodiment, pet store 102 and small breeders 103 can sign a consignment agreement 105, which allows pet store 102 to sell pets 101 on behalf of small breeders 103. Once the offspring are produced by small breeders 103, the offspring can be profiled as discussed further below and then sold by pet store 102. In another embodiment, pets 101 can be sourced from hobby breeders 104. In this embodiment, pet store 102 and each hobby breeder 104 can sign a hobby breeder’s program 106 agreement, which permits pet store 102 to facilitate the breeding process of pets 101 bought by hobby breeder 104. Facilitating the breeding process can include determining a suitable match for a specific pet 101 to produce a desired offspring, which is further detailed below. In such embodiments, each pet owner 107 can be a supplier of pets 101 for pet store 102. Furthermore, a percentage of the profit made from selling the pets can be paid to the small breeder or hobby breeder.

For purposes of this disclosure, pet store 102 can range between 5,000 to 10,000 square feet. As such, pets can be displayed in glass display with no more than 4-6 pets per display. Furthermore, each pet store 102 can have an interaction room where a prospective pet owner can spend time with pet 101 before purchasing. This can ensure that future pet owners are comfortable and will be satisfied with the pet their trying to purchase. Each pet store 102 can also have an office space for a veterinarian, and veterinarian staffs such as veterinarian technician and pet caregivers. Thus, a licensed veterinarian can be on site at each pet store 102. This can ensure that there is always a veterinarian to provide consultation and services at pet store 102. In one embodiment, pet care givers can stay and watch pet store 102 around the clock or 24/7. In another embodiment, pet care givers can watch the pets offsite via camera placed within pet store 102. Such services can give a good impression to customers that pet’s needs are taken care off and that pets are treated properly.

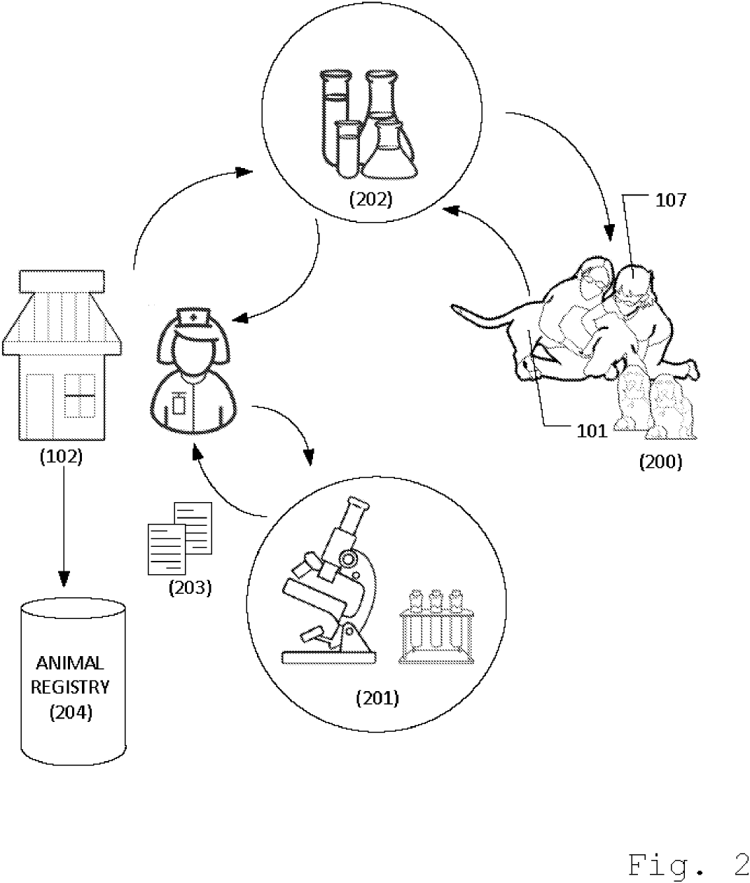

FIG. 2 illustrates profiling pet 101 on pet supply chain system 100. To ensure that only desirable animals 101 are being commercialized in pet store 102, the proper profiling of each pet 101 can also be monitored. Initially, pets 101 can be sourced from small or intermediate breeders who engage with pet store 102. As such, once the animals from small breeders 103 produces one or more offspring 200, each offspring can be collected and can be commercialized as pets 101 on pet store 102. In one embodiment, pet store 101 can first identify genetic characteristics 300 of pets 101 to be sold in the market by performing a genetic test 201 for each offspring 200.

Genetic test 201 can be performed on a genetic sample 202 of an animal. Genetic sample 202 can be materials such as blood, hair, skin, amniotic fluid, or other tissue taken from an animal. Furthermore, genetic testing 201 can be done to look for changes in an animal’s genes or changes in the amount, function, or structure of key proteins coded by specific genes. Some examples of genetic test 201 can include but are not limited to chromosome studies, DNA studies, and biochemical genetic studies. For purposes of this disclosure, chromosomes are parts of the cells that contain an animal’s genes. As such, genes are parts of DNA passed down from a parent to offspring. Chromosomes studies can be done when an offspring is born with multiple birth defects in order to look for specific changes in the order of the chromosome material linked with the defects. Further, DNA studies can be used to identify changes in DNA sequence or chromosome structure. Tests for cancer susceptibility genes are usually done by DNA studies. Lastly, biochemical genetic studies involve the study of enzymes in the body of the animal that may be abnormal in some way.

The result of genetic testing 201, which is known as genetic test result 203, can confirm or rule out a suspected genetic condition of an animal. Moreover, genetic test result 203 can help determine if an animal has a chance of developing or passing a genetic disorder to their offspring. Thus, genetic test result 203 can help in producing offspring 200 with a desired genetic characteristic, which will be discussed further below.

After receiving genetic test result 203, offspring 200 can then be registered by pet store 102 into one of one or more animal club registries 204. This is to ensure pet owners 107 that pets 101 being sold at pet store 102 maintains a breed standard. Animal club registries 204 can be a club that maintains breed standards and/or records pedigrees of an animal. An example of animal club registry 204 is a kennel club registry which can include but is not limited to the American Kennel Club, True Bully Kennel Club (TBKC), and Bully Breed Kennel Club (BBKC). As such, animal club registries 204 can manage a list of adult purebred animals and lists of offspring born to purebred parents.

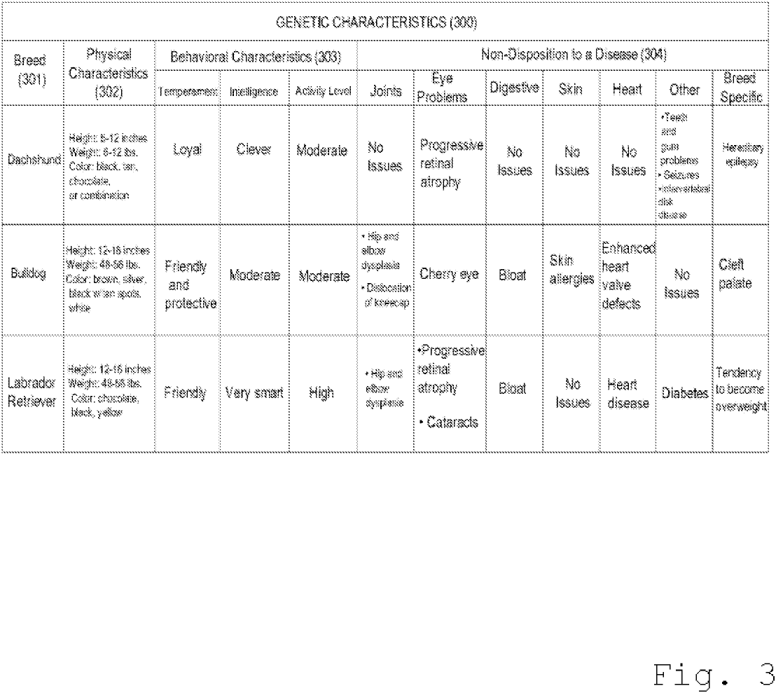

FIG. 3 illustrates an example of genetic characteristics 300 of dog breeds. Genetic characteristics 300 can be traits that an animal inherits from its patents. Genetic characteristics 300 can also relate to genetic test result 203 that was taken from pet 101 before being commercialized in pet store 102. In one embodiment, genetic characteristics 300 can be associated with the animal’s breed 301. Some examples of genetic characteristics 300 that can be passed on to offspring 200 can include but is not limited to physical characteristics 302, behavioral characteristics 303, and a non-disposition to a disease 304. Using dog breeds as an example, physical characteristics 302 can include but is not limited to animal’s height, weight, size, color, markings, body coverings (e.g., fur, hair, coat), body, etc.

Further, genes can also contribute to behavioral characteristics 303 of an animal. Although each dog or animal can have their individual personality, there are still some behavioral characteristics 303 that can be attributed to different breed types. As illustrated in FIG. 3, Dachshund breed can be loyal, clever and can have moderate activity level while bulldogs can be friendly and protective and can have moderate intelligence and activity level. And Labrador Retriever can be friendly, very smart, and can have a high activity level.

Non-disposition to a disease 304 can be the genetic susceptibility of an animal to a disease. Though most breeds can have a high probability of developing a breed-specific diseases, each individual animal can have their own medical history 805 and each animal may have a low or reduced chance in developing a disease, which can be associated to each animal’s genes. As shown in FIG. 3, some dog breed can have an increased likelihood of developing a particular disease based on the animal’s genetic makeup. For example, Dachshund have no known issues with their joint, digestive system, skin, and heart. But Dachshund can be susceptible to eye problem such as progressive retinal atrophy, can have hereditary epilepsy, and can have other diseases such as teeth and gum problems, seizures, and intervertebral disk disease. Bulldogs can be susceptible with joint problems such as hip and elbow dysplasia and dislocation of knee cap, can also have cherry eye problem, skin allergies, enhanced heart valve defects, and cleft palate. While Labrador Retriever breed can have no issues with skin but can be susceptible to joint problems such as hip and elbow dysplasia, can also have eye problem such as cataracts and progressive retinal atrophy. Labrador Retriever can also have digestive issue such as bloat, can have heart disease, susceptible to diabetes, and has tendency to become overweight.

Identifying genetic characteristics 300 of each offspring 200 can be very useful, as such process can help pet supply chain 100 identify a good match amongst the network of pets 101 within the supply chain system.

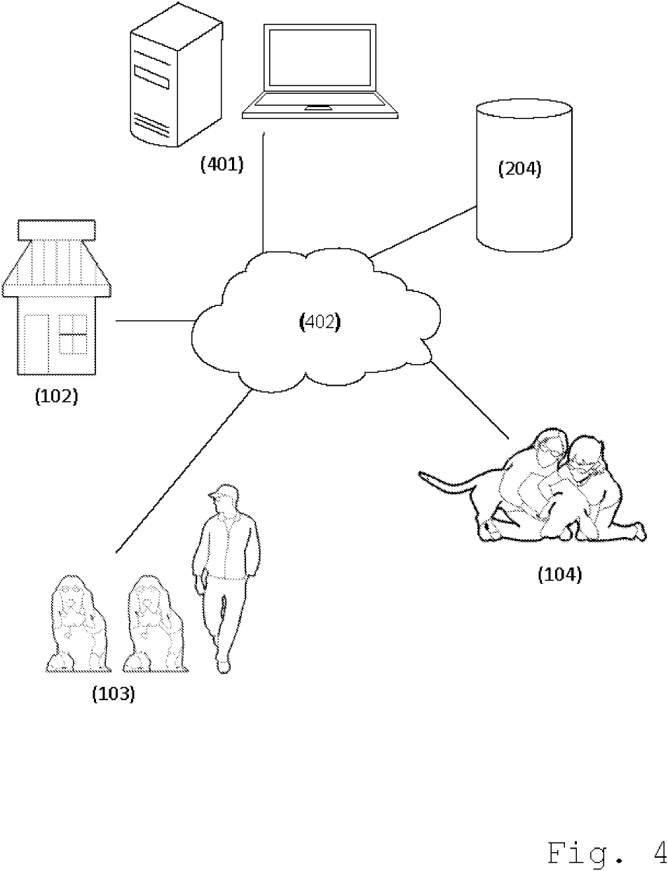

FIG. 4 illustrates another embodiment of pet supply chain system 100 further comprising a pet application 401. In one embodiment, pet supply chain system 100 can use pet application 401 to manage data information of small breeders 103, hobby breeders 104, and pets 101. In one embodiment, pet application 401 can be a website. Pet application 401 can comprise business logic for a server or other device. In one embodiment, pet system application 401 can be device agnostic, wherein the application can be machine independent and can be configured to work with various system. In one embodiment, pet application 401 can be a centralized online system configured to manage and monitor a supply chain. In such embodiment, pet stores 102, small breeders 103, and hobby breeders 104 can be interconnected through pet application 401 via network 402. In this embodiment, each pet store 102 can be a facility wherein pets 101 can be displayed, housed, bred, cared for, and sold. Each pet store 102 can use pet application 401 to market pets and provide services to pets. As such, each pet store 102 can be a one-stop shop for all pet needs. Network 402 can be a local area network (LAN), a wide area network (WAN), a piconet, or a combination of LANs, WANs, or piconets. One illustrative LAN is a network within a single business. One illustrative WAN is the Internet. In a preferred embodiment, network 402 will comprise the Internet.

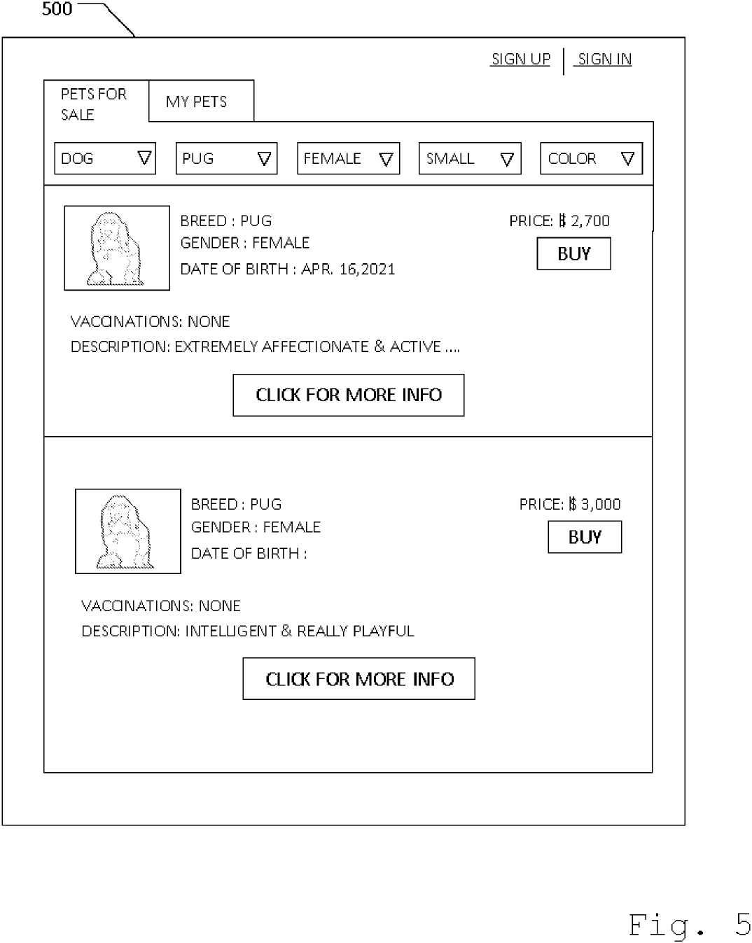

FIG. 5 illustrates a screenshot of an embodiment of pet application 401. In one embodiment, main screen 500 can comprise list of pets 101 that are being sold in pet store 102. In one embodiment, any user can access main screen 500 without logging in or registering on pet application 401. As such, user can browse through the list of pets 101 being sold on pet application 401. In a scenario wherein user would like to buy or sell pet 101 on pet application 401, user would be required to sign-up on the application. Registering on pet application 401 can also allow the user to purchase pet 101 in staggard payment. Further in one embodiment, display on main screen 400 can vary depending on an account type of the user who is logged in.

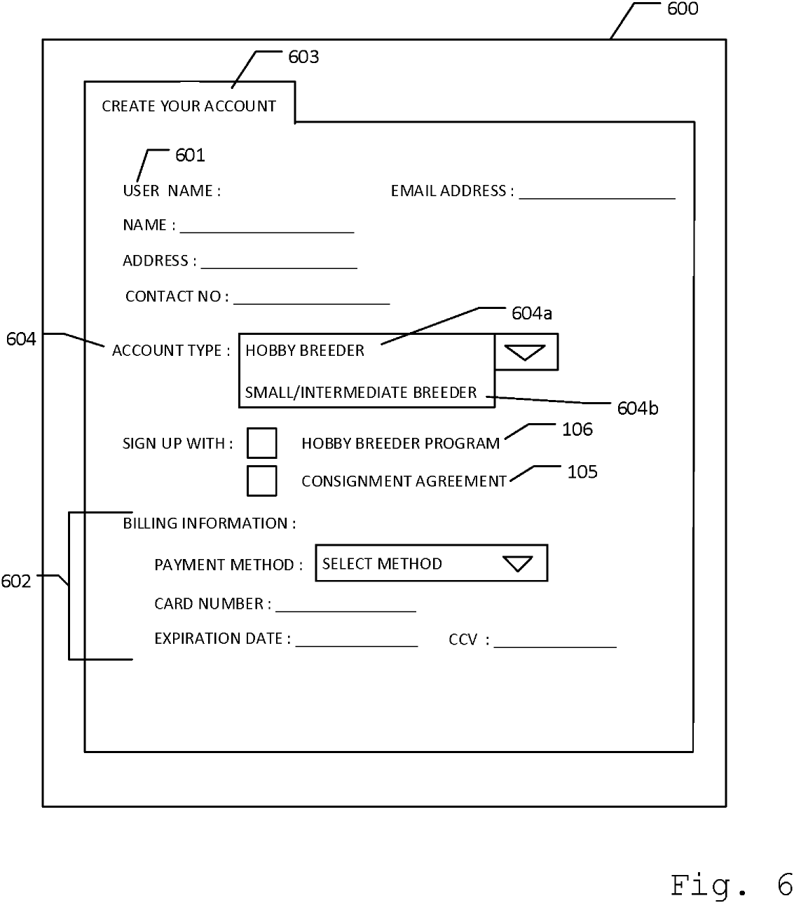

FIG. 6 illustrates a sign-up screen 600. Sign-up screen can allow a user to be registered on pet application 401. In this screen, user can be required to supply unique credentials such as to username 601, password, name, email address, contact information, delivery address, and billing information 602. Thus, registering on sign-up screen 600 can relate to creating a user account 603 on pet application 401. Username 601 can be a unique alphanumeric character that associates a user’s account with pet owner 107. Furthermore, user can select an account type 604 being created. Household individuals that can be interested in buying pets 101 can set up a hobby-breeder account 604a, while small or intermediate breeder who may be interested in buying and/or selling pets 101 through pet application 401 can select a small breeder account 604b. Users who select hobby-breeder account 604a and bought pet 101 from pet application 401 can choose to sign-up with hobby-breeder program 106 while, a user who has small breeder account 604b can sign-up with consignment agreement 105. Thus, pet owner 107 can receive a payment for each successful transaction made by selling the pets 101 from pet store 102.

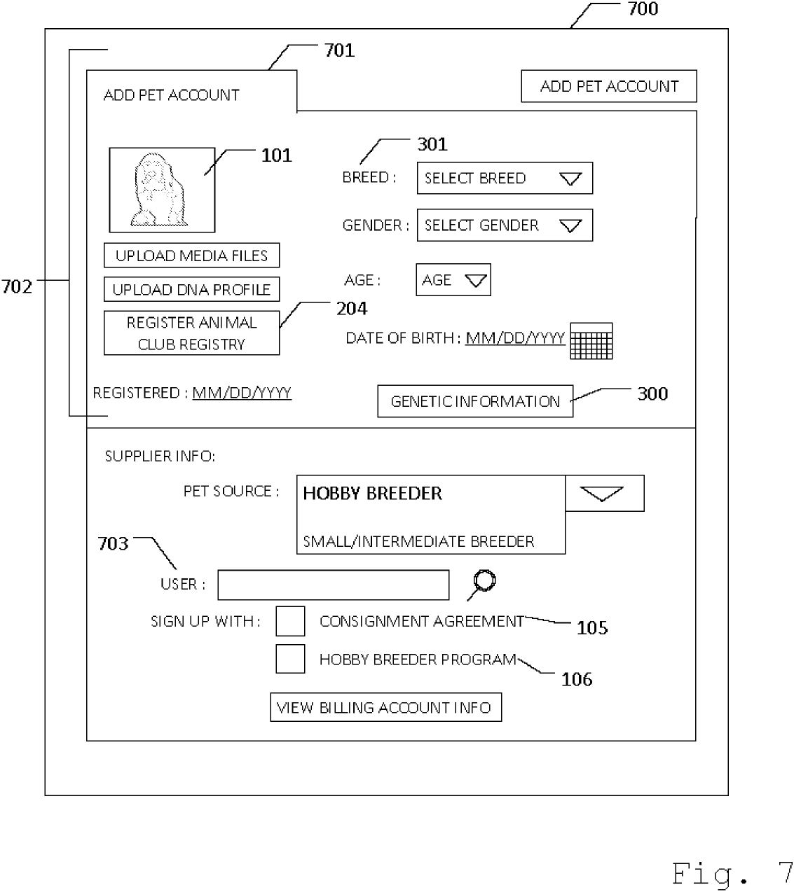

FIG. 7 illustrates an add-pet screen 700. In one embodiment, each pet 101 displayed on main screen 400 can be added by a pet store personnel. In one embodiment, only users with an admin account can access add-pet screen 700. In such embodiment once offspring 200 is supplied to pet store 102 the pet store personnel can register the offspring as pet 101 on pet application 401. Adding pet 101 on the screen can be related to creating a pet account 701 on pet application 401. Further, add-pet screen 700 can allow the personnel to fill-up a pet profile 702 and genetic characteristics 300 of pet 101 that is being registered on the application. Pet profile 702 can include information on pet 101 that can include but is not limited to, a unique identification number, breed 301, registration date, birth date, sex, age, photos and videos. Moreover, the pet store personnel can also register pet 101 to one of animal club registries 204. In one embodiment, clicking register can automatically send pet profile 702 of pet 101 to one of animal club registries 204. Once registration is granted, kennel club registration certificate can be sent to pet application 401 and stored on the corresponding pet account. In one embodiment, certification can be provided for offspring 200 if results of the offspring’s genetic testing 201 have the characteristics 300 that is within one more predetermined thresholds. In one embodiment, animal registry 204 can be an automated service wherein registration certificate can be delivered to pet owner’s 107 registered delivery address. As such, pet owner does not have to go through the pet store 102 to pick up his pet’s certificate. In one embodiment, registration certificate can include pet’s 106 paw print.

Further in one embodiment, add-pet screen 700 can also comprise username field 703. Username field 703 can allow the pet personnel to associate pet 101 with username 601 that is associated with a pet owner’s user account 603. In one embodiment, by selecting a specific username 601 on username field 703, the application can determine whether the selected user has signed up with either consignment agreement 105 or hobby-breeder program 106. In this embodiment, user account 603 associated to username 601 can receive a portion of agreed payment once pet 101 associated with pet account 701 is sold. In such embodiment, payment can be automatically sent to the associated billing information 602 of user account 603.

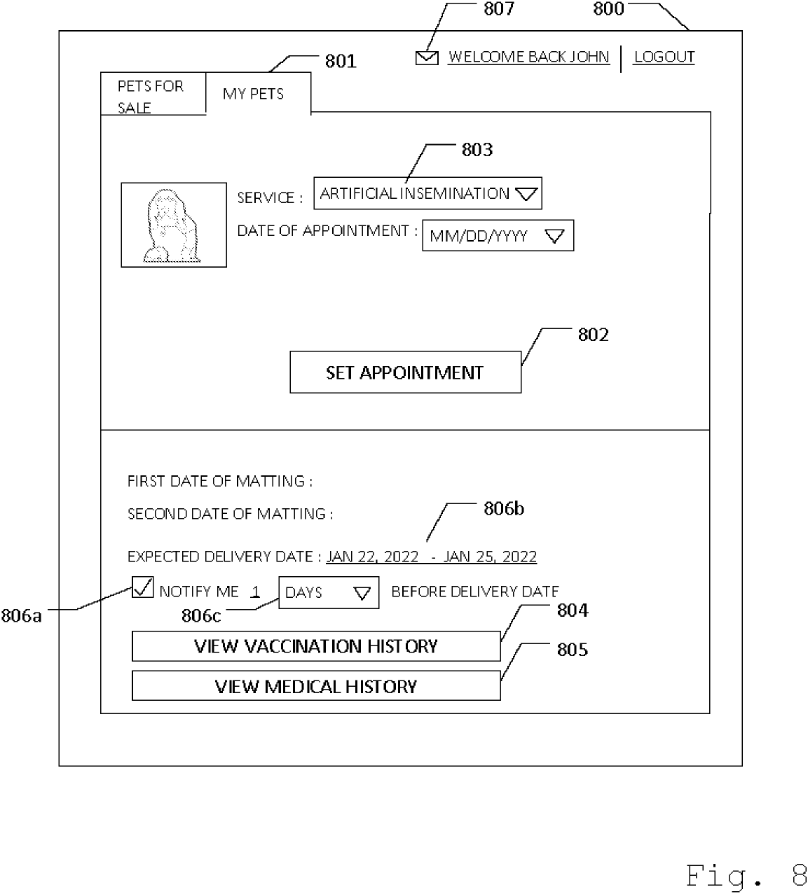

FIG. 8 illustrates a pet owner’s screen 800 comprising a pets list 801. User’s pets list 801 can display pets 101 bought by the user that is currently logged in. In one embodiment, pet owner’s screen 800 can allow user to schedule an appointment request 802 for one or more pet services 803. Furthermore, pet owner’s screen 800 can allow user to view pet’s 101 vaccination information 804 and medical history 805. Vaccination information 804 can contain information such as type of vaccine, date of shots, and schedules of next vaccination. Medical history 805 can contain information of animal’s health, which can include but are not limited to animal’s surgeries, illnesses, and results of laboratory tests. In some embodiment, medical history 805 can also include information about medicines used for each illness, and other health information such as animal’s diet and exercise. In one embodiment, pet owner’s screen 800 can also comprise notification settings such as a notify-me widget 806a, a duration widget 806b, and a frequency widget 806c. Notify-me widget, duration widget, and frequency widget can each be an interactive tool, which can be displayed as a link, check box, calendar button, dropdown box, or a button.

In an example scenario, pet owner 107 can click on notify-me widget 806a to be notified for the next vaccination date of his pet 101. In one embodiment, pet owner can set the duration of the notification through duration widget 806b and can select the frequency of the notification through frequency widget 806c. In one embodiment, pet owner 107 can choose to be notified through the contact information such as e-mail address or contact number, which was set up during the sign-up registration on pet application 401. In another embodiment, pet application 401 can send a notification alert 807 to users on the scheduled appointment date and time, when there is a request for breeding permissions, and when a match is found for the pet owner’s pet 101.

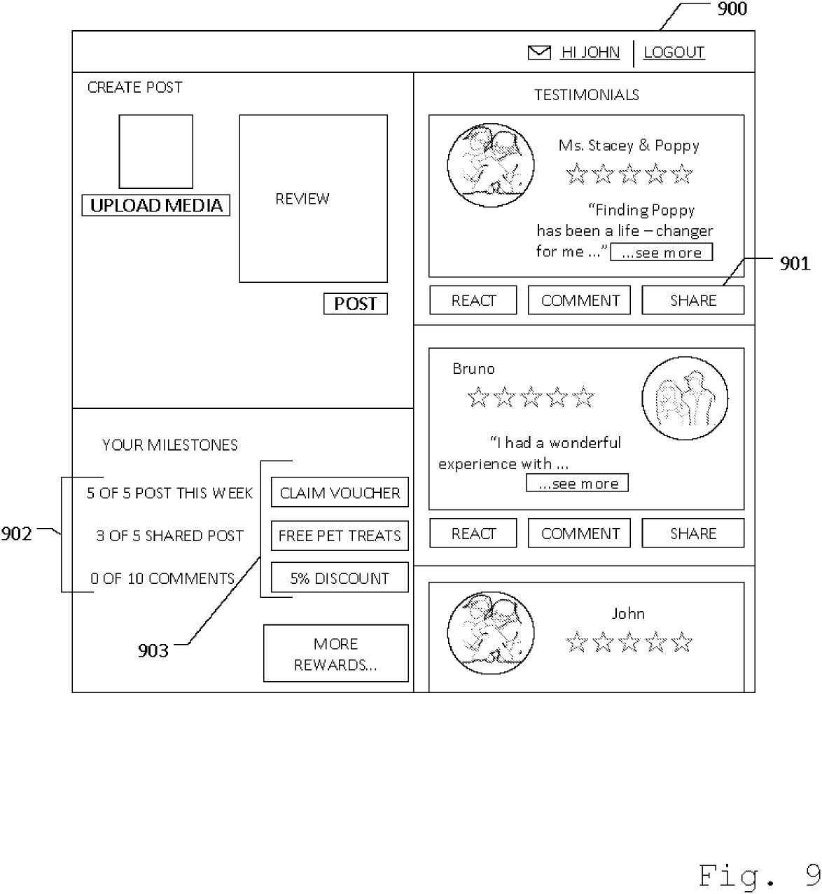

FIG. 9 illustrates a social network page 900 of pet application 401. In one embodiment, pet owners can create contents or post a testimonial related to owning pet 101 that is bought from pet application 401. In such embodiment, pet application 401 can feature the stories of pets 101 and pet owners 107 on social network page 900. As such, users accessing pet application 401 can view, share, comment, or react to the content posted on pet application 401. In another embodiment, contents created on pet application can also be shared and posted in different social media platforms through a share-to-social media widget 901, which can include but is not limited to Facebook, Instagram, TikTok, or YouTube. Further in one embodiment, pet owners who create contents, posts, comments or establish any social engagement on pet application 401 can be incentivized on pet application 401. In an example embodiment, pet application 401 can show one or more tasks 902, which can be done by the users. As such, each task 902 that is completed can earn the user different rewards 903 such as vouchers, discount coupons, merchandise, pet food, or free or discounted pet services. This feature can encourage pet owners to create content, and share their experience through social media.

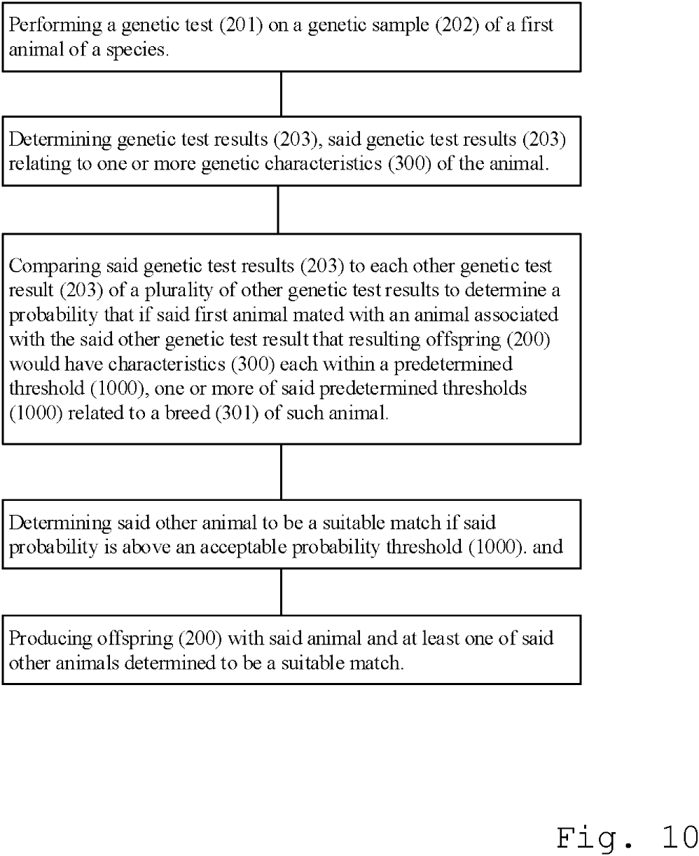

FIG. 10 illustrates an exemplary method for producing an animal breed. In one embodiment, a contract, which is consignment agreement 105 can be established between pet store 102 with small and intermediate sized breeders 103. In another embodiment, a contract, which is hobby-breeder program 106 can also be established by pet store 102 with hobby breeder 104. In such embodiments, signing up with the contract can be done online through pet application 401. In an embodiment wherein pet application 401 can be a website, each pet owner 107 can first be registered on the website to sign the contract. Once signed, genetic test 201 can be performed on a genetic sample 202 of a first animal of a species. As an example, pet store 102 can assign their inhouse veterinarian to gather genetic sample 202 from a first pet 101a. This can then provide a pet store personnel the information needed to store pet profile 702 and genetic characteristics 300 for first pet 101a. In another embodiment, an at-home kit or genetic test kit can be sent to pet owners 107 for collection of genetic samples 202 to be performed by the breeder or pet owner. In one embodiment, pet profile 702 and genetic characteristics 300 can also be used to register first pet 101a on one of animal club registries 204. Then, the genetic sample can be sent back to either pet store 102 or pet application 401 to be analyzed. The information gathered can then be stored in data store of pet application 401. After gathering of genetic samples 202 genetic test result 203 for first pet 101a can be determined, genetic test result 203 can be related to one or more genetic characteristics 300 of the animal, Genetic test results 203 for first pet 101a can then be compared to each of the other genetic test result of a plurality of other genetic test results to determine a probability that if first pet 101a is mated with an animal associated with the other genetic test result, that resulting offspring 200 would have characteristics 300 each within predetermined threshold 1000.

Pet application 401 can then determine other pet 101 to be a suitable match with first pet 101a if probability is above an acceptable probability threshold. After determining a suitable match for first pet 101a, an offspring 200 can then be produced with first pet 101a and at least one of other pets 101 that was determined to be a suitable match. Once supply of pets 106 are obtained, pets 106 can then be displayed and sold in pet store 102.

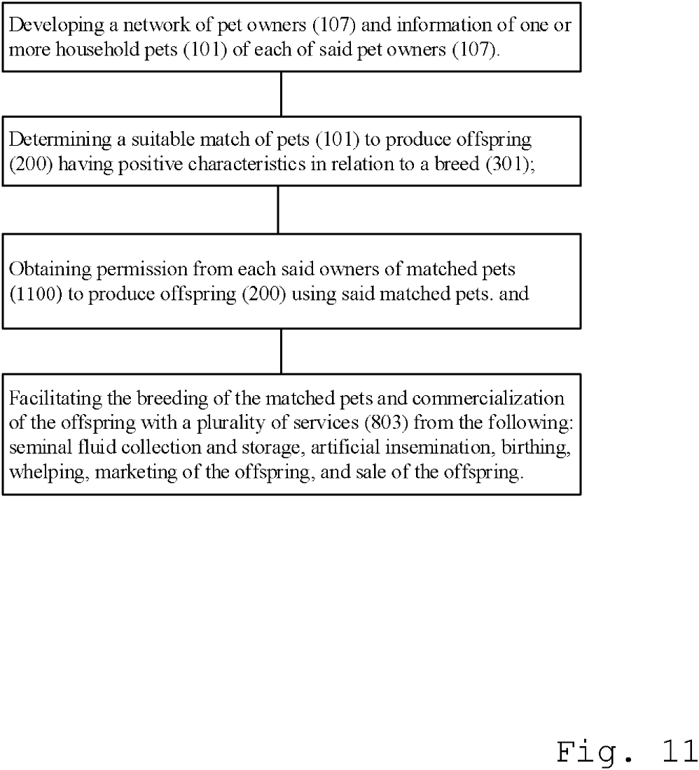

FIG. 11 illustrates an exemplary method for integrating a network of household pet owners into pet supply chain 100. Any individual can access pet application 401 to participate in pet supply chain 100. However only registered individuals can buy, sell, and use pet services 803 that are offered through pet application 401. Furthermore, only the animals that are registered on pet application 401 can be sold in pet store 102. As such, each animal being marketed in pet store 102 and pet application 401 should first be registered by a pet store personnel. To do so, the pet store personnel can first gather the animal’s information in order to store the data as pet account 701. Pet account 701 can include information on pet’s 106 profile 702, and genetic characteristics 300. In a scenario wherein the animal is already registered, other pet information can be stored within the animal’s pet account 701. Other pet information can include but is not limited to vaccination information 804 and medical history 805. As such, pet application 401 can maintain a network of registered users such as pet hobby breeders 104 and small breeders 103 non pet supply chain 100. Furthermore, pet application 401 can maintain information of each pet 101 of each pet owner 107. Each pet account 701 can comprise information that can be used to determine a suitable match for each pet 101 within pet supply chain 100. Thus, pet application 401 can determine a suitable match of pet 101 to produce offspring 200 that have positive characteristics in relation to a specific breed 301. The application can then associate suitable matches for each pet 101 that is within pet supply chain 100. After pet application 401 determines a suitable match of pets that can produce the desired offspring, the application can then obtain permission from each pet owners 107 of one or more matched pets 1100.

In an example scenario wherein pet store 102 wants to find a match for a first pet owner’s 107a with a first pet 101a that is a Bulldog breed 301, pet application 401 can query data store 204 to determine other pets 101 within pet application 401 that is within a predetermined threshold 1001. The predetermined threshold can be related to breed 301 of pet 101. In this example scenario wherein predetermined threshold 1000 on pet application 401 can be set to color of the animal such as “black with tan spots” and animal that has less likely to develop a heart disease, the application can start to query genetic characteristics 300 of each pet account 701 whose breed 301 is “Bulldog”. The application can query through other pet accounts 701 to find a suitable mate for first pet 101a that has “black with tan spots” color with minimal or no history of heart disease on medical history 805, and whose non-disposition to a disease for heart disease is “low”. The results of such query can create matched pets 1100 on pet application 401 and matched pets 1100 can be associated with first pet 101a. Matched pets 1100 can comprise other pet accounts 701 that have the positive characteristics described on predetermined threshold 1000.

In one embodiment, pet application 401 can notify first pet owners 107a that a suitable match for their pet 101 exists. Pet application 401 can then solicit first owner 107a to have their first pet 101a participate in producing offspring 200. In one embodiment, pet application 401 can send notification alert 807 to the owner to inform them that their pets 101 is a suitable match for another pet 101. In another embodiment, pet application 401 can send a message via email or SMS (short message service) to ask permission from the owner to breed their pets. Further in another embodiment, the owner can be notified through notification alert 807 on pet application 401. Once the first owner agrees, the application can then get permission from other owner’s whose pets 101 is a suitable match for first pet 101a. After receiving permission from both owners, pet store 102 can start with facilitate the breeding of matched pets 1100 and then eventually start the commercialization of the pet’s offspring 200, which can be further discussed below.

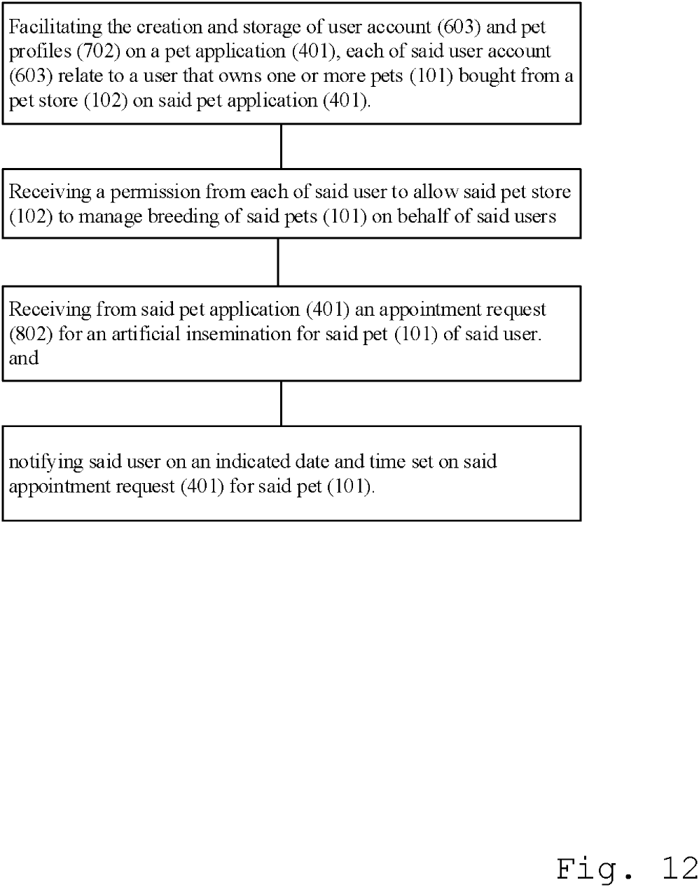

FIG. 12 illustrates an exemplary method for providing scheduled pet services to pet 101. In an embodiment wherein user signs up with hobby-breeder program 106, pet store 102 can help in facilitating the breeding process of pet 101. In an example scenario wherein pet 101 can be a mature male dog, pets 106 participating with hobby-breeder program 106 can be scheduled for the collection of seminal fluid. Such pet service appointment request 802 can be scheduled through pet application 401. As such after setting appointment request 802, pet owner 107 can drop the pet by the pet store and have a pet store personnel take the samples from pet 101. The seminal fluid can be collected, frozen, and stored by pet store personnel. After getting the pet service, pet 101 can then be brought home by the pet owner. The seminal fluid collected can then be used for artificial insemination by pet store 102 on a suitable female pet 101 once a suitable match is found. Furthermore, the pay that pet owner can receive from male pet 101 can be based on the stud’s ability to produce quality offspring.

In another example scenario wherein pet 101 is a female dog, on pet’s 101 2^{nd} heat pet owner 107 can set up an artificial insemination appointment through pet application 401. After the artificial insemination is done, pet owner 107 can then return home with his pet 101. In one embodiment, pet application 401 can suggest to pet owner a follow-up pet service appointment scheduled on the pet’s labor day. In such embodiment, pet application 401 can determine the estimated labor date of pet 101. In one embodiment, pet application 401 can notify pet owner 107 within the application. In another embodiment, pet application 401 can send a message on pet owner’s registered contact information. Then on pet’s labor day, pet 101 can be assisted by the veterinarian on the pet store’s birthing and whelping facility. In one embodiment, the owner can bring pet 101 to pet store 102 on its labor day. In another embodiment, pet store 102 can schedule a pickup of pet 101 from pet owner’s home. Pet 101 and its offspring can then remain within pet store’s whelping facility for six to eight weeks, and can be under the care of the store’s veterinary technicians. Pet account 701 for each offspring 200 can be created and added to data store of pet application 401 and the offspring can be moved to the showroom to be sold and displayed. Pet 101 can then be picked up by the owner or dropped by the pet store to the pet owner’s residence through the delivery service of pet store 102. A percentage of a completed sale for each pet 101 sold can then be paid or sent to billing information 602 of pet owner 107. Further, the delivery and/or pickup services can be offered to pet owners 107 who signed the contract with pet store 102. The convenience of pickup and delivery service for pets before and/or after labor can encourage more pet owners 107 to sign up with the contract.

The flowcharts of FIG. 10, FIG. 11, and FIG. 12 shows the functionality and operation of an implementation of portions of pet application 401. If embodied in software, each block can represent a module, segment, or portion of code that comprises program instructions to implement the specified logical function(s). The program instructions can be embodied in the form of source code that comprises human-readable statements written in a programming language or machine code that comprises numerical instructions recognizable by a suitable execution system such as server processor in a computer system or other system. The machine code can be converted from the source code, etc. If embodied in hardware, each block can represent a circuit or a number of interconnected circuits to implement the specified logical function(s). Although the flowcharts of FIG. 10, FIG. 11, and FIG. 12 show a specific order of execution, the order of execution can differ from what is depicted. For example, the order of execution of two or more blocks can be rearranged relative to the order shown. Also, two or more blocks shown in succession in FIG. 10, FIG. 11, and FIG. 12 can be executed concurrently or with partial concurrence. In addition, any number of counters, state variables, warning semaphores, or messages might be added to the logical flow described herein, for purposes of enhanced utility, accounting, performance measurement, or providing troubleshooting aids, etc. All such variations are within the scope of the present disclosure.

Also, any logic or application described herein that comprises software or code, including pet application 401, can be embodied in any computer-readable storage medium for use by or in connection with an instruction execution system such as, server processor in a computer system or other system. The logic can comprise statements including instructions and declarations that can be fetched from the computer-readable storage medium and executed by the instruction execution system.

In the context of the present disclosure, a “computer-readable storage medium” can be any medium that can contain, store, or maintain the logic or application described herein for use by or in connection with the instruction execution system. The computer-readable storage medium can comprise any one of many physical media, such as electronic, magnetic, optical, electromagnetic, infrared, or semiconductor media. More specific examples of a suitable computer-readable storage medium can include, but are not limited to, magnetic tapes, magnetic floppy diskettes, magnetic hard drives, memory cards, solid-state drives, USB flash drives, or optical discs. Also, the computer-readable storage medium can be a random access memory (RAM), including static random access memory (SRAM), dynamic random access memory (DRAM) or magnetic random access memory (MRAM). In addition, the computer-readable storage medium can be a read-only memory (ROM), a programmable read-only memory (PROM), an erasable programmable read-only memory (EPROM), an electrically erasable programmable read-only memory (EEPROM), or other type of memory device.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. An improved method of producing an animal of a particular breed comprising the steps:
performing a genetic test on a genetic sample of a first animal of a species;
determining genetic test results, said genetic test results relating to one or more genetic characteristics of the animal;
comparing said genetic test results to each other genetic test result of a plurality of other genetic test results to determine a probability that if said first animal mated with an animal associated with the said other genetic test result that resulting offspring would have characteristics each within a predetermined threshold, one or more of said predetermined thresholds related to a breed of such animal;
determining said other animal to be a suitable match if said probability is above an acceptable probability threshold; and
producing offspring with said animal and at least one of said other animals determined to be a suitable match.

2. The method of claim 1 wherein said one or more genetic characteristics associated with said breed comprises physical characteristics.

3. The method of claim 1 wherein said one or more genetic characteristics associated with said breed comprises behavioral characteristics.

4. The method of claim 1 wherein said one or more genetic characteristics associated with said breed comprise a non-disposition to a disease.

5. The method of claim 1 wherein said first animal is an animal of a first pet owner and said other genetic test results are from other animals belonging to other pet owners.

6. The method of claim 5 wherein said first pet owner and said other pet owners provide samples from their pets at a location of a retail establishment or using an at-home kit from said retail establishment.

7. The method of claim 11 wherein producing offspring comprises notifying said first pet owner that said suitable match exists and soliciting said first pet owner to have said first animal participate in producing said offspring.

8. The method of claim 1 wherein producing said offspring comprises using artificial insemination.

9. The method of claim 1 further comprising registering said offspring on a registry based on the breed of such offspring.

10. The method of claim 1 further comprising performing offspring genetic testing on each of the offspring.

11. The method of claim 10 further comprising the step of providing a certification for each of said offspring if results of said offspring genetic testing have said characteristics within said predetermined thresholds.

12. The method of claim 1 wherein said animal is a dog.

13. The method of claim 1 wherein said genetic test comprises a DNA study.

14. The method of claim 1 wherein said genetic test comprises a chromosomal study.

15. The method of claim 1 wherein said genetic test comprises a biochemical study.
